(19) United States
(12) Patent Application Publication
(10) Pub. No.: US 2018/0303311 A1
(43) Pub. Date: Oct. 25, 2018

(54) ENDOSCOPE APPARATUS, ENDOSCOPE SYSTEM AND REPORT GENERATION METHOD

(71) Applicant: OLYMPUS CORPORATION, Tokyo (JP)

(72) Inventor: Tsuyoshi FURUHATA, Tokyo (JP)

(73) Assignee: OLYMPUS CORPORATION, Tokyo (JP)

(21) Appl. No.: 15/957,299

(22) Filed: Apr. 19, 2018

(30) Foreign Application Priority Data
Apr. 25, 2017 (JP) 2017-086183

(51) Int. Cl. A61B 1/00; A61B 1/04; G06F 3/0488; H04N 5/374

(52) U.S. Cl. CPC A61B 1/00004; A61B 1/00039; A61B 1/0002; H04N 5/374; A61B 1/04; G06F 3/0488; A61B 1/00045

(57) ABSTRACT

An endoscope apparatus includes: a processor including hardware, wherein the processor is configured to acquire information about an object that can be controlled by a rotation assisting tool configured to cause an object including a rotating body to rotate, from the rotation assisting tool; and a first storage configured to store reference information corresponding to the information about the object acquired by the processor.

### CROSS-REFERENCE TO RELATED APPLICATION

This application is based upon and claims the benefit of priority from the prior Japanese Patent Application No. 2017-86183 filed on Apr. 25, 2017; the entire contents of which are incorporated herein by reference.

### BACKGROUND

### 1. Technical Field

The present invention relates to an endoscope apparatus for examining a subject, an endoscope system and a report generation method.

### 2. Background Art

Conventionally, endoscope apparatuses making it possible to, by inserting an elongated insertion portion into a body cavity, observe an organ and the like in the body cavity and, if necessary, perform various therapeutic treatments using a treatment instrument inserted in a treatment instrument channel have been widely used. Further, in an industrial field also, industrial endoscope apparatuses are widely used for observation and examination of internal cracks, corrosion and the like of a boiler, a turbine, an engine, a chemical plant and the like.

As is well known, a turbine is a rotary machine for expanding working fluid to take out thermal energy of the working fluid as mechanical work. In a power plant, for example, a lot of gas turbines, steam turbines and the like are used. In order to perform examination of abrasion, damage and the like for turbine blades (hereinafter simply referred to as blades) of the gas turbines and the steam turbines, the industrial endoscope apparatuses are used.

In such examination for a steam turbine, a distal end portion of an endoscope insertion portion (hereinafter simply referred to as an insertion portion) is inserted from an access point (an access port) provided on the steam turbine in order to examine all blades. Then, a method is often used in which an examiner inspects the blades one by one while manually causing the blades to rotate little by little in a state where the distal end portion of the insertion portion is arranged at a position where the blades can be observed. In the method, since the examiner manually rotates the blades little by little, time and effort are required. Therefore, a method is proposed in which, at the time of examining blades, a rotation assisting tool for causing the blades to automatically rotate is used in order to improve examination efficiency (for example, Japanese Patent Application Laid-Open Publication No. 2007-113412).

### SUMMARY

An endoscope apparatus of an aspect of the present invention includes: a processor including hardware, wherein the processor is configured to acquire information about an object that can be controlled by a rotation assisting tool configured to cause an object including a rotating body to rotate, from the rotation assisting tool; and a first storage configured to store reference information corresponding to the information about the object acquired by the processor.

Further, an endoscope system of an aspect of the present invention includes an endoscope apparatus including: a rotation assisting tool configured to cause an object including a rotating body to rotate; and a first storage configured to, when acquiring information about an object that can be controlled by the rotation assisting tool from rotation assisting tool, store reference information corresponding to the information about the object acquired by the processor.

Further, a report generation method of an aspect of the present invention includes: acquiring information about an object that can be controlled by a rotation assisting tool configured to cause an object including a rotating body to rotate, from the rotation assisting tool; and generating a report with an image of the object picked up by an image sensor and reference information corresponding to the acquired information about the object arranged in the same report.

### BRIEF DESCRIPTION OF THE DRAWINGS

FIG. 1 is a diagram showing an overall configuration of an endoscope apparatus according to a first embodiment;

FIG. 2 is a diagram showing an example of control target information held by a first storage 36 of a body portion 3;

FIG. 3 is a diagram showing an example of reference information associated with each piece of control target information;

FIG. 4 is a diagram showing an example of examination image storage destinations associated with each piece of control target information;

FIG. 5 is a flowchart showing an example of a flow of an examination image recording process;

FIG. 6 is a diagram showing an example of examination images recorded by the recording process of FIG. 5;

FIG. 7 is a diagram showing another example of the examination images recorded by the recording process of FIG. 5;

FIG. 8 is a flowchart showing an example of a flow of a report generating process;

FIG. 9 is a diagram showing an example of a report generated by the report generating process of FIG. 8;

FIG. 10 is a flowchart showing an example of a flow of a report generating process;

FIG. 11 is a diagram showing an example of a report generated by the report generating process of FIG. 10;

FIG. 12 is a diagram showing another example of the report generated by the report generating process of FIG. 10;

FIG. 13 is a diagram showing another example of the report generated by the report generating process of FIG. 10;

FIG. 14 is a diagram showing reference information associated with each control target; and

FIG. 15 is a flowchart showing an example of a flow of a report generating process.

### DETAILED DESCRIPTION OF THE PREFERRED EMBODIMENT(S)

Embodiments of the present invention will be described below with reference to drawings.

Note that, in each drawing used in the description below, reduced scale may be different for each component in order to show the component in a recognizable size on the drawing. That is, the present invention is not limited to the number of components, shapes of the components, a ratio of sizes of the components, relative positional relationships among the respective components shown in the drawings.

### First Embodiment

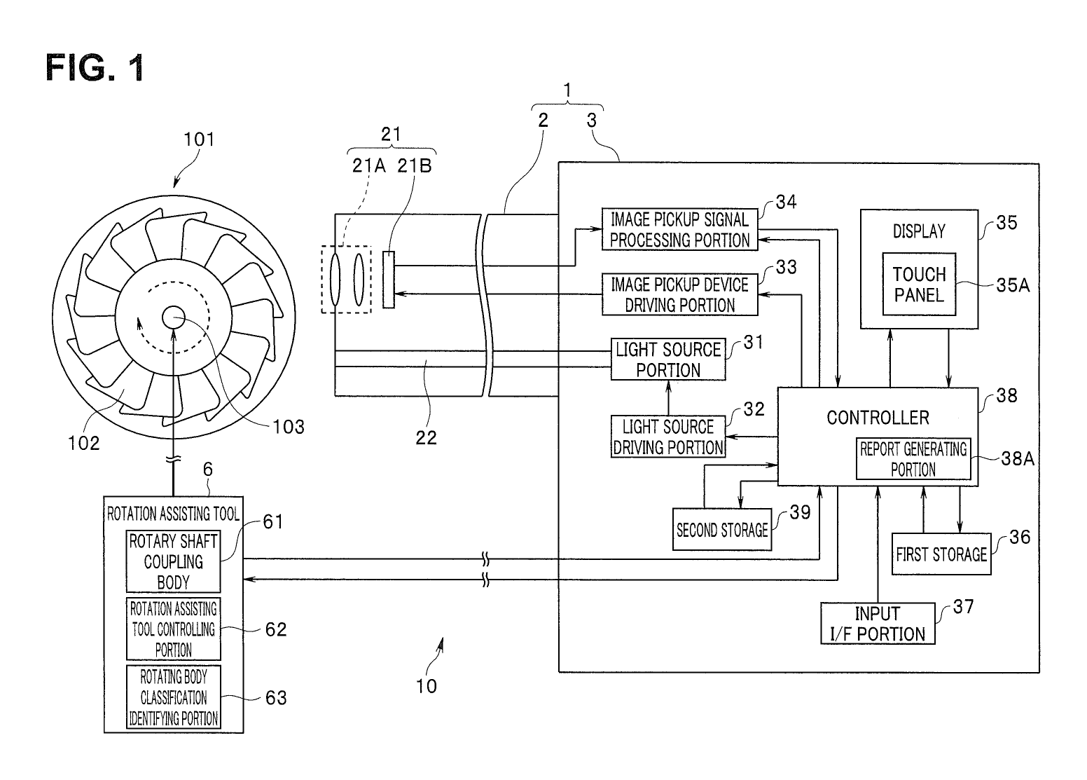

FIG. 1 is a diagram showing an overall configuration of an endoscope apparatus according to a first embodiment.

As shown in FIG. 1, an endoscope system 10 of the present embodiment is configured including an endoscope apparatus 1 and a rotation assisting tool 6. The endoscope apparatus 1 is configured including an insertion portion 2, which is, for example, formed being provided with an elongated shape which can be inserted into a casing of a steam turbine from an access port and flexibility, and a body portion 3 connected to a proximal end portion of the insertion portion 2.

At a distal end portion of the insertion portion 2, an image sensor 21 is provided which is configured to be capable of picking up an image of turbine blades (hereinafter briefly referred to as blades) 102 of a turbine body 101 which is an object provided in the casing of the steam turbine. Further, inside the insertion portion 2, a light guide 22 for guiding illuminating light supplied from the body portion 3 to the distal end portion of the insertion portion 2 to emit the illuminating light to the blades 102, which are an examination region, is provided.

Note that, hereinafter, description will be made on an assumption that the turbine body 101 is configured being provided with the plurality of blades 102 and a turbine rotary shaft 103. Further, hereinafter, the description will be made on an assumption that the turbine body 101 is configured to be capable of causing the plurality of blades 102 to rotationally move according to rotation of the turbine rotary shaft 103.

The image sensor 21 is configured including an objective lens unit 21A and an image pickup device 21B.

The objective lens unit 21A is configured being provided with one or more lenses for forming an image of reflected light from an examination region (an object) illuminated by illuminating light emitted through the light guide 22.

The image pickup device 21B is configured, for example, being provided with a CCD or a CMOS. Further, the image pickup device 21B is configured to be driven according to an image pickup device driving signal outputted from the body portion 3. Further, the image pickup device 21B is configured to pick up an image of reflected light image-formed by the objective lens unit 21A to generate an image pickup signal, and output the generated image pickup signal to the body portion 3.

The body portion 3 is configured so that the body portion 3 can be connected to the rotation assisting tool 6 provided outside the endoscope apparatus 1, via a signal cable or a communication cable. Further, the body portion 3 is configured including a light source portion 31, a light source driving portion 32, an image pickup device driving portion 33, an image pickup signal processing portion 34, a display 35, a first storage 36, an input I/F (interface) portion 37, a controller 38 and a second storage 39.

The light source portion 31 is configured, for example, being provided with an LED or a lamp. Further, the light source portion 31 is configured to be turned on or off according to a light source driving signal outputted from the light source driving portion 32. Further, for example, the light source portion 31 is configured to supply, for example, white light with a light quantity corresponding to a light source driving signal outputted from the light source driving portion 32, to the light guide 22 as illuminating light.

The light source driving portion 32 is configured, for example, being provided with a light source driving circuit. Further, the light source driving portion 32 is configured to generate and output a light source driving signal for causing the light source portion 31 to be driven, according to control of the controller 38.

The image pickup device driving portion 33 is configured, for example, being provided with an image pickup device driving circuit. Further, the image pickup device driving portion 33 is configured to generate and output an image pickup device driving signal for causing the image pickup device 21B to be driven, according to control of the controller 38.

The image pickup signal processing portion 34 is configured, for example, being provided with a signal processing circuit. Further, the image pickup signal processing portion 34 is configured to generate endoscopic image data by performing predetermined signal processing for an image pickup signal outputted from the image pickup device 21B and sequentially output the generated endoscopic image data to the controller 38, according to control of the controller 38. That is, the image pickup signal processing portion 34 is configured being provided with a function of generating and sequentially outputting images of the turbine body 101 picked up by the image sensor 21 as an image generating portion.

The display 35 is configured, for example, being provided with a liquid crystal panel. Further, the display 35 is configured to display an image corresponding to display image data outputted from the controller 38, on a display screen. Further, the display 35 is configured including a touch panel 35A configured to detect a touch operation on a GUI (graphical user interface) button or the like displayed on the display screen and output an instruction corresponding to the detected touch operation to the controller 38.

The first storage 36 is configured, for example, being provided with a storage circuit such as a memory. Further, the first storage 36 is configured to be capable of storing still image data and movie data corresponding to endoscopic image data generated by the image pickup signal processing portion 34. Further, in the first storage 36, a program used for control of each portion of the endoscope apparatus 1 by the controller 38, and the like is stored. Further, the first storage 36 is configured so that data and the like generated according to an operation of the controller 38 is appropriately stored.

The input UF portion 37 is configured being provided with switches and the like capable of giving an instruction corresponding to an input operation by a user to the controller 38. Further, the input I/F portion 37 is configured to be capable of inputting rotation control information which is information used for control of the rotation assisting tool 6 by the controller 38 (to be described later), according to an operation by the user.

The controller 38 as a processor configured with hardware is configured to perform control for the light source driving portion 32, the image pickup device driving portion 33 and the image pickup signal processing portion 34 based on an instruction given according to a touch operation on the touch panel 35A and/or an instruction given according to an operation of the input I/F portion 37. Further, the controller 38 is configured to, based on rotation control information inputted according to an operation of the input I/F portion 37 and rotation information outputted from a rotation assisting tool controlling portion 62 to be described later, perform setting and control with regard to rotational movement of the plurality of blades 102 for the rotation assisting tool controlling portion 62. Further, the controller 38 is configured to be capable of generating display image data in which GUI buttons and the like are superimposed on image data such as endoscopic image data outputted from the image pickup signal processing portion 34, and outputting the display image data to the display 35. Further, the controller 38 is configured to be capable of encoding endoscopic image data outputted from the image pickup signal processing portion 34 into still image data such as JPEG data and movie data such as MPEG4 data and storing the still image data and the movie data into the first storage 36. Further, the controller 38 is configured to be capable of, based on an instruction given according to an operation of the touch panel 35A or the input I/F portion 37, reading image data (still image data and movie data) stored in the first storage 36, generating display image data corresponding to the read image data and outputting the display image data to the display 35. Further, the controller 38 is configured to perform predetermined image processing such as color space conversion, interlace/progressive conversion and gamma correction for the display image data to be outputted to the display 35. Further, the controller 38 is configured including a report generating portion 38A configured to create, according to an instruction operation from the user, a report on which an image of an object picked up by the image sensor 21 (an inspection image) and reference information about the object are arranged together.

The rotation assisting tool 6 is configured to be capable of being connected to the controller 38 of the body portion 3 via a signal cable or a communication cable. Further, the rotation assisting tool 6 is configured including a rotary shaft coupling body 61, a rotation assisting tool controlling portion 62 and a rotating body classification identifying portion 63. Further, the rotation assisting tool 6 is configured to be capable of being connected to the turbine rotary shaft 103 of the turbine body 101 via the rotary shaft coupling body 61. Further, the rotation assisting tool 6 is configured to be capable of making settings for an operation of the rotary shaft coupling body 61 according to setting and control of rotational movement of the plurality of blades 102 performed by the controller 38 of the body portion 3. The user can perform control of the rotation assisting tool 6, for example, by using the touch panel 35A or the input OF portion 37 provided on the body portion 3. Note that the user may perform control of the rotation assisting tool 6 by a remote controller (not shown) connected to the rotation assisting tool 6.

The rotary shaft coupling body 61 is configured, for example, being provided with a gear and the like. Further, the rotary shaft coupling body 61 is configured to be capable of generating rotational force by being rotated under a parameter set according to a rotation assisting tool control signal outputted from the rotation assisting tool controlling portion 62 and causing the plurality of blades 102 to rotationally move by supplying the generated rotational force to the turbine rotary shaft 103.

The rotation assisting tool controlling portion 62 is configured, for example, being provided with a control circuit and a drive circuit. Further, the rotation assisting tool controlling portion 62 is configured to generate and output a rotation assisting tool control signal for performing setting and control of the rotation assisting tool 6 according to control of the controller 38 of the body portion 3. Further, the rotation assisting tool controlling portion 62 is configured to, for example, based on a rotation state of the rotary shaft coupling body 61, acquire rotation information which is information capable of identifying a current rotation position of the plurality of blades 102 which are rotationally moved by the rotary shaft coupling body 61 and transmit the acquired rotation information to the body portion 3.

The rotating body classification identifying portion 63 is configured to, based on an instruction from the controller 38 of the body portion 3, identify a classification of an object connected to the rotation assisting tool 6 and transmit the identified classification of the object to the controller 38. Based on whether the classification of the object has been transmitted from the rotating body classification identifying portion 63, the controller 38 can judge whether or not the rotation assisting tool 6 is connected to the turbine body 101 which is the object.

The first storage 36 of the body portion 3 holds control target information which is information about turbines which the rotation assisting tool 6 can control, that is, the rotation assisting tool 6 can assist rotation of. The controller 38 can acquire the control target information based on a classification of an object transmitted from the rotating body classification identifying portion 63.

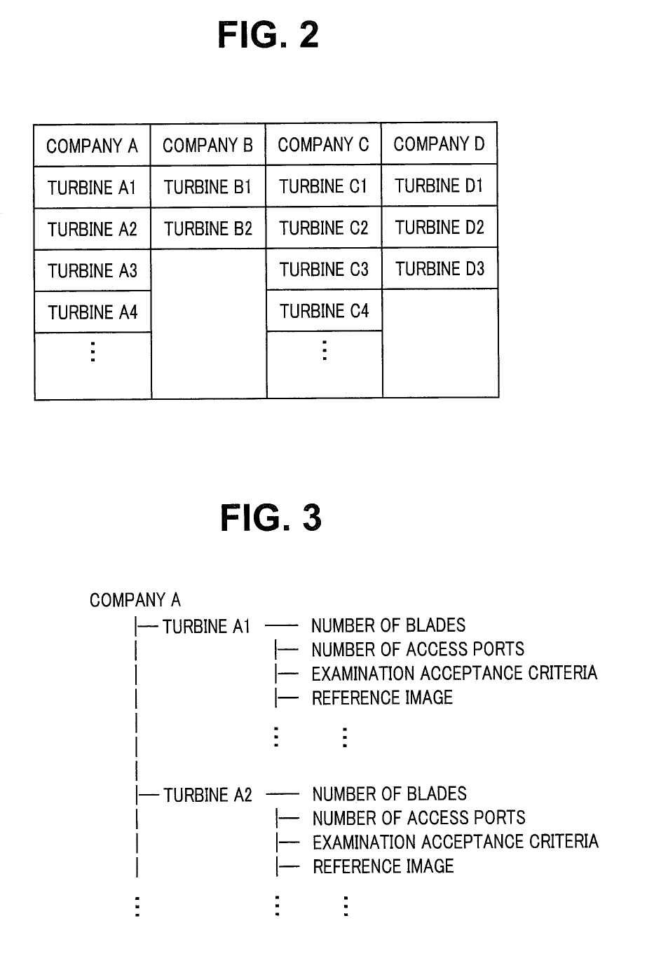

FIG. 2 is a diagram showing an example of the control target information held by the first storage 36 of the body portion 3. As shown in FIG. 2, for example, for turbines of Company A, the rotation assisting tool 6 can assist rotation of turbines A1, A2, A3, A4 . . . . On the other hand, for turbines of Company B, the rotation assisting tool 6 can assist rotation of only turbines B1 and B2. The controller 38 of the body portion 3 is configured to acquire a classification of a currently controlled object from the rotation assisting tool 6.

The first storage 36 of the body portion 3 holds reference information associated with each piece of control target information. FIG. 3 is a diagram showing an example of the reference information associated with each piece of control target information. As shown in FIG. 3, the reference information includes at least pieces of information of the number of blades, the number of access ports, examination acceptance criteria and a reference image, and the pieces of information are associated with each piece of control target information. Note that the reference information is not limited to the information of the number of blades, the number of access ports, the examination acceptance criteria and the reference image. For example, images photographed in the past, an image showing an examination procedure and the like may be associated with each piece of control target information as the reference information.

When acquiring control target information from the rotation assisting tool 6 based on information about a classification of an object, the controller 38 reads reference information corresponding to the control target information from the first storage 36. Then, when an instruction to record an image is given from the user, the controller 38 arranges an image of the object picked up by the image sensor 21 (an inspection image) and the reference information read from the first storage 36 together and stores the image and the reference information into the second storage 39 as one still image (an examination image). At this time, the image of the object picked up by the image sensor 21 (the inspection image) is stored into the second storage 39. The reference information arranged together with the inspection image may include all of the number of blades, the number of access ports, the examination acceptance criteria and the reference image or may include, for example, only the reference image.

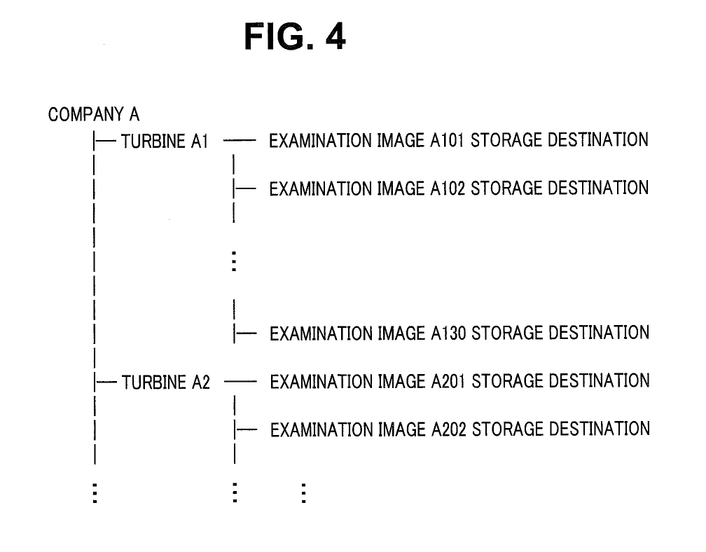

Further, the second storage 39 holds information about an examination image storage destination associated with each piece of control target information. FIG. 4 is a diagram showing an example of examination image storage destinations associated with each piece of control target information.

For example, if the number of blades of the turbine A1 of Company A is thirty, the user is required to photograph images of the thirty blades 102. Therefore, thirty folders of an examination image A101 storage destination to an examination image A130 storage destination are associated with a folder of the turbine A1 of Company A in the second storage 39. The controller 38 stores examination images of the thirty blades into the folders of the examination image A101 storage destination to the examination image A130 storage destination. Thus, the controller 38 acquires images corresponding to a circumference of an object, which is a rotating body, and stores the images corresponding to the circumference of the object into a same folder (the folder of the turbine A1). Then, for example, when the object is changed from the turbine A1 to the turbine A2, images of the turbine A2 are stored into a folder different from the folder of the turbine A1 (a folder of the turbine A2). That is, the controller 38 stores images of an object including a first rotating body (for example, the turbine A1) and images of an object including a second rotating body (for example, the turbine A2) into different folders. The examination images stored into the examination image A101 storage destination to the examination image A130 storage destination become examination images of a same group, and, at the time of generating a report to be described later, the examination images are attached to one report as examination images of the same group.

The same group is a range for which examination can be performed at a time without removing the rotation assisting tool 6 from the turbine body 101. That is, the same group is a range for which the turbine is caused to be rotated once (360 degrees) by the rotation assisting tool 6. For example, when the examination target is changed from the turbine A1 to the turbine A2 of Company A, detachment and attachment of the rotation assisting tool 6 occurs, and, therefore, the turbine A1 and the turbine A2 correspond to different groups. The rotation assisting tool controlling portion 62 detects whether a turbine was caused to rotate 360 degrees, for example, based on information from an encoder (not shown) attached to the turbine rotary shaft 103. If the turbine was caused to rotate 360 degrees, the rotation assisting tool controlling portion 62 notifies the controller 38 of the body portion 3 that the turbine rotated once. For example, the controller 38 displays that the turbine rotated once on the display 35 to inform the user of completion of examination.

The controller 38 determines, by acquiring information about a currently connected object from the rotation assisting tool 6, whether the object is fitted to the rotation assisting tool 6 or not. If the information about the object can be acquired from the rotation assisting tool 6, the controller 38 determines that the object is fitted to the rotation assisting tool 6. If the information about the object cannot be acquired from the rotation assisting tool 6, the controller 38 determines that the object has been detached from the rotation assisting tool 6. Then, if the information about the object changes, the controller 38 changes a folder for storing images of the object.

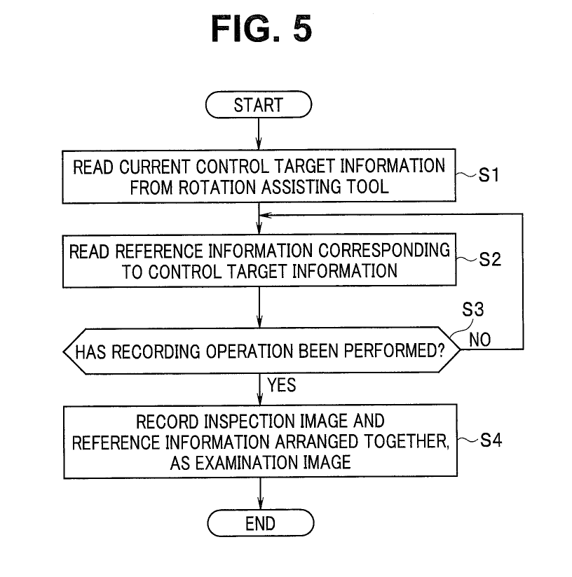

Next, a specific operation and the like of an examination image recording process of the endoscope apparatus 1 will be described with reference to FIGS. 5 to 7.

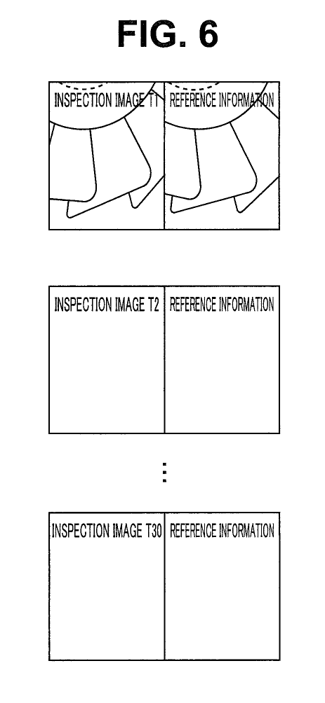

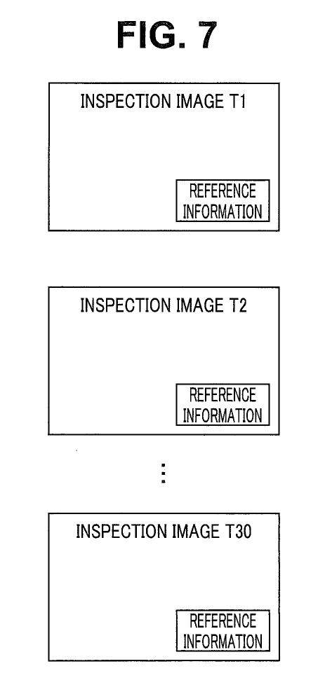

FIG. 5 is a flowchart showing an example of a flow of the examination image recording process; FIG. 6 is a diagram showing an example of examination images recorded by the recording process of FIG. 5; and FIG. 7 is a diagram showing another example of the examination images recorded by the recording process of FIG. 5.

First, the controller 38 reads current control target information from the rotation assisting tool 6 at step S1 and proceeds to step S2. At step S2, the controller 38 reads a reference image corresponding to the control target information from the first storage 36. At step S3, the controller 38 judges whether a recording operation has been performed or not. If judging, at step S3, that a recording operation has not been performed, the controller 38 proceeds to step S2 and repeats a similar process. On the other hand, if judging, at step S3, that the recording operation has been performed, the controller 38 proceeds to step S4. At step S4, the controller 38 records an inspection image and the reference information arranged together, as an examination image, and ends the recording process.

By the above process, the endoscope apparatus 1 can acquire recorded information about an image of an object.

By the examination image recording process, the controller 38 arranges an inspection image T1 and reference information together as shown in FIG. 6 and records the inspection image T1 and the reference information into the second storage 39 which is a second storage, as one examination image. Note that FIG. 6 shows an example in which a reference image is arranged together with the inspection image T1 as the reference information. Each time the recording process of FIG. 5 is executed, an examination image on which each of inspection images T1, T2, . . . and reference information are arranged together is recorded into the second storage 39. At this time, each of the inspection images T1, T2, . . . is stored into the second storage 39.

Further, the controller 38 may arrange a reference image in a predetermined area of each of the inspection images T1, T2, . . . in a picture-in-picture format as shown in FIG. 7 and record the reference image and the inspection image into the second storage 39 as an examination image. The user can create a report using examination images recorded in this way.

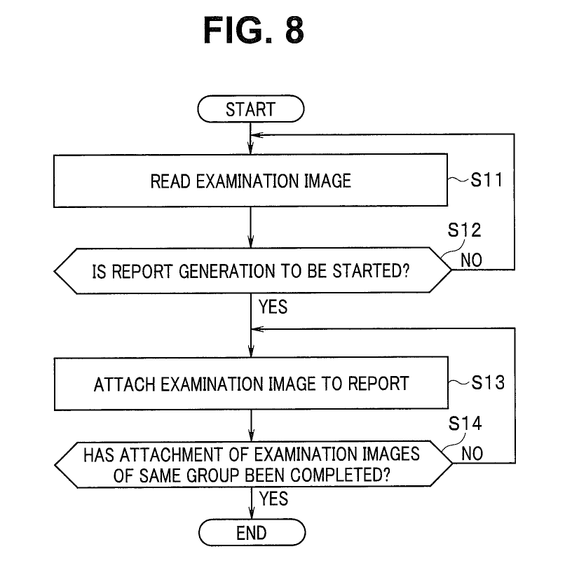

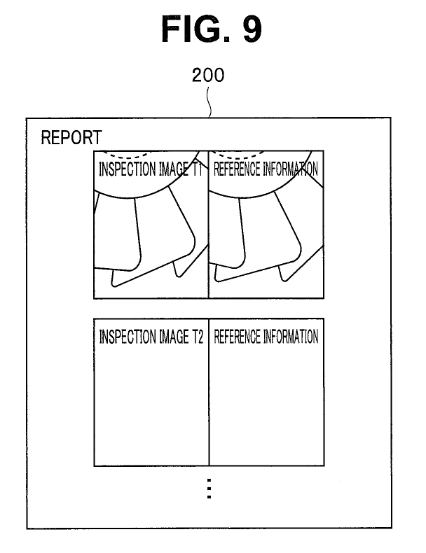

Next, a specific operation and the like of a report generating process of the endoscope apparatus 1 of the first embodiment will be described with reference to FIGS. 8 and 9.

FIG. 8 is a flowchart showing an example of a flow of the report generating process. FIG. 9 is a diagram showing an example of a report generated by the report generating process of FIG. 8. Note that the report generating process shown in FIG. 8 is executed by the report generating portion 38A of the controller 38.

First, the report generating portion 38A reads an examination image from the second storage 39 at step S11 and proceeds to step S12. At step S12, the report generating portion 38A judges whether an instruction to start report generation has been given or not. For example, the instruction to start report generation is given by the user using the touch panel 35A or the input I/F portion 37.

If judging that an instruction to start report generation has not been given, the report generating portion 38A proceeds to step S11 and repeats a similar process. On the other hand, if judging that an instruction to start report generation has been given, the report generating portion 38A proceeds to step S13 and attaches the examination image to a report.

Next, at step S14, the report generating portion 38A judges whether attachment of examination images of a same group has been completed or not. If judging that attachment of the examination images of the same group has not been completed, the report generating portion 38A proceeds to step S13 and repeats a similar process. On the other hand, if judging that attachment of the examination images of the same group has been completed, the report generating portion 38A ends the report generating process.

To a report 200 generated by the report generating process, all the examination images of a turbine belonging to the same group are attached as shown in FIG. 9. That is, the report generating portion 38A generates the report 200 obtained by arranging images corresponding to a circumference of an object and pieces of reference information corresponding to information about the object acquired by the controller 38 on the same report. For example, if the turbine A1 includes thirty blades 102, thirty examination images are attached to the report 200. Since, in each of the examination images attached to the report 200, an inspection image obtained by the endoscope apparatus 1 and a reference image corresponding to control target information are arranged together, the user can easily compare the inspection image and the reference image.

### Second Embodiment

Next, a second embodiment will be described. In the first embodiment, the controller 38 attaches reference information to an inspection image to generate an examination image at the time of performing a recording process, and the report generating portion 38A attaches the examination image to a report at the time of generating the report.

In comparison, in the second embodiment, at the time of performing a recording process, control target information is attached to an inspection image to generate an examination image; and, at the time of generating a report, the control target information is read, and reference information is attached to the report.

When a recording operation is performed, the controller 38 attaches control target information to an inspection image, for example, in an Exif file format and records the control target information and the inspection image into the second storage 39 as an examination image. As a result, the endoscope apparatus 1 can acquire recorded information about an image of an object.

When a report generation instruction is given by the user, the report generating portion 38A reads control target information attached to an inspection image. Then, the report generating portion 38A reads reference information corresponding to the read control target information from the first storage 36 and attaches the reference information to a report.

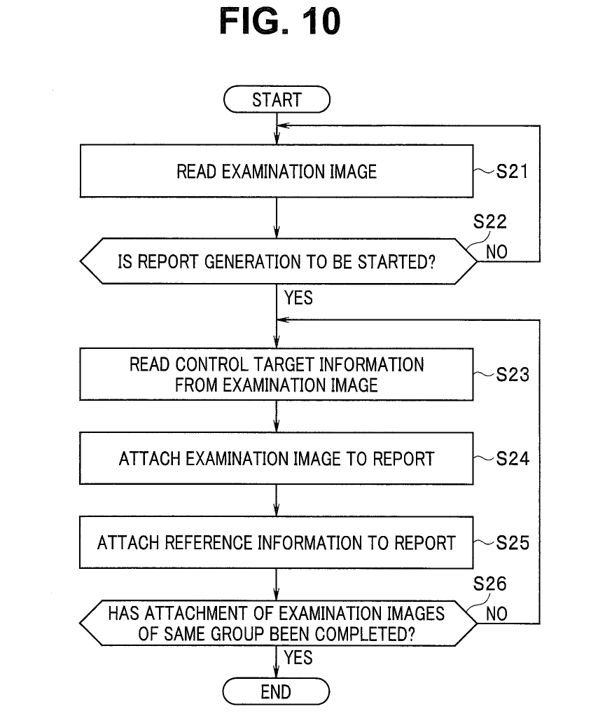

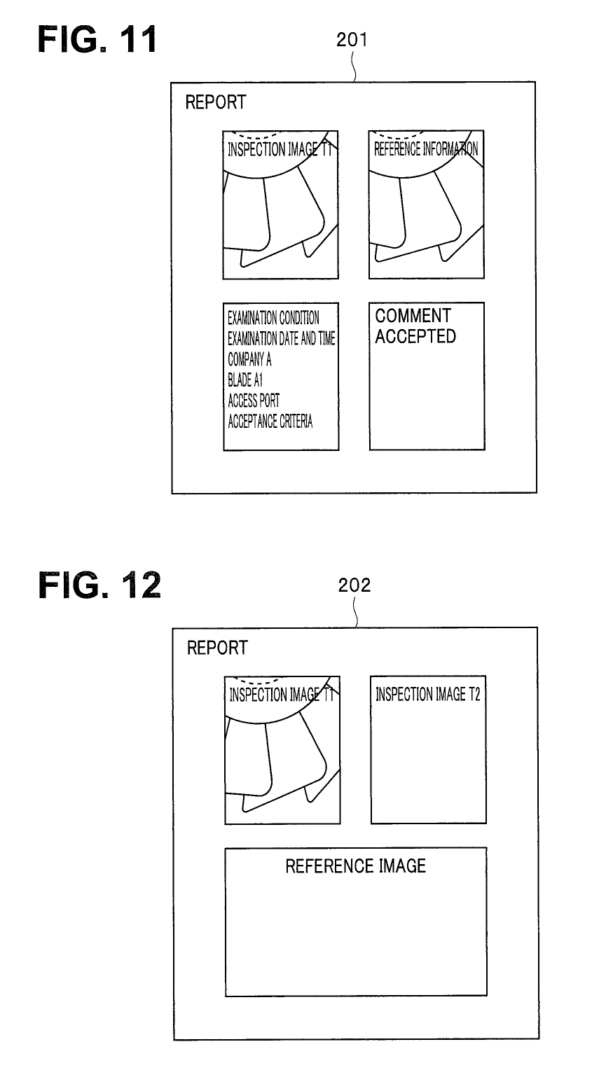

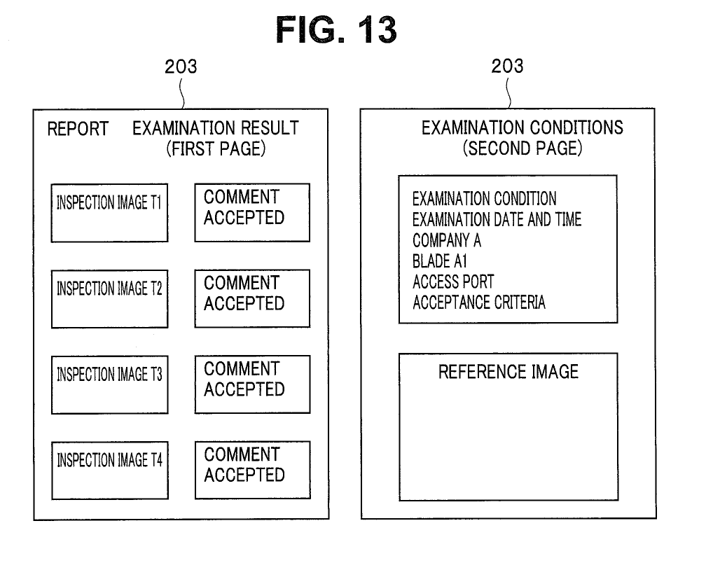

Next, a specific operation and the like of a report generating process of the endoscope apparatus 1 of the second embodiment will be described with reference to FIGS. 10 to 13. FIG. 10 is a flowchart showing an example of a flow of the report generating process; FIG. 11 is a diagram showing an example of a report generated by the report generating process of FIG. 10; and FIGS. 12 and 13 are diagrams showing other examples of the report generated by the report generating process of FIG. 10.

First, the report generating portion 38A reads an examination image from the second storage 39 at step S21 and proceeds to step S22. At step S22, the report generating portion 38A judges whether an instruction to start report generation has been given or not. For example, the instruction to start report generation is given by the user using the touch panel 35A or the input I/F portion 37.

If judging that an instruction to start report generation has not been given, the report generating portion 38A proceeds to step S21 and repeats a similar process. On the other hand, if judging that an instruction to start report generation has been given, the report generating portion 38A proceeds to step S23 and reads control target information from the examination image.

At step S24, the report generating portion 38A attaches the examination image to a report. Then, at step S25, the report generating portion 38A attaches reference information corresponding to the read control target information to the report.

At step S26, the report generating portion 38A judges whether attachment of examination images of a same group has been completed or not. If judging that attachment of the examination images of the same group has not been completed, the report generating portion 38A proceeds to step S23 and repeats a similar process. On the other hand, if judging that attachment of the examination images of the same group has been completed, the report generating portion 38A ends the report generating process.

By such a report generating process, a report 201 to which an inspection image T1, a reference image and reference information such as an examination condition are attached is generated as shown in FIG. 11. The report 201 is provided with a comment field in which the user can input, for example, an examination result such as “accepted” and “not accepted”.

Further, the report generating portion 38A may generate a report 202 on which one reference image is attached together for a plurality of inspection images T1 and T2 as shown in FIG. 12. In this case, the report generating portion 38A attaches, for example, an image of a whole turbine provided with a plurality of blades to the report 202 as the reference image.

Further, the report generating portion 38A may generate a report 203 configured with a plurality of pages as shown in FIG. 13. For example, the report generating portion 38A generates an examination result in which a plurality of inspection images and a comment for each of the plurality of inspection images are inputted on a first page, and generates examination conditions such as an examination condition and a reference image on a second page. Note that the report generating portion 38A may attach to the report 203 an inspection image T1 by rotating orientation of the inspection image T1 so that a gravity direction of the inspection image T1 corresponds to a gravity direction of the reference image.

By confirming a report generated in this way, the user can easily confirm an examination result and whether an examination has been correctly performed.

### Modification

Next, a modification of the second embodiment will be described.

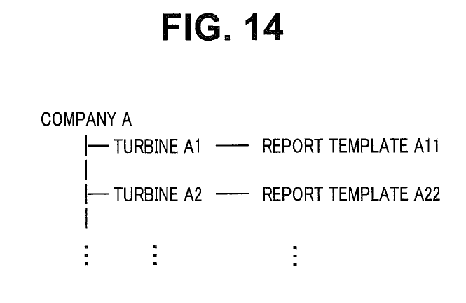

FIG. 14 is a diagram showing reference information associated with each control target. As shown in FIG. 14, in the modification, information about a report template is provided as the reference information about each control target.

When a report generation instruction is given by the user, the report generating portion 38A reads control target information attached to an inspection image. The report generating portion 38A reads a report template (a template file) from reference information corresponding to the read control target information. For example, if the inspection image shows blades of the turbine A1, the report generating portion 38A reads a report template All and attaches inspection images of a same group to generate a report. Thus, the report generating portion 38A generates a report using a template file corresponding to information about an object acquired by the controller 38.

In a report template, an image as a reference (a reference image), images photographed in the past, a design image, a guide image showing an examination procedure and the like are shown in advance. Therefore, only by reading a report template corresponding to control target information and attaching an inspection image to the read report template at the time of generating a report, the reports 201 to 203 as shown in FIGS. 11 to 13 can be generated.

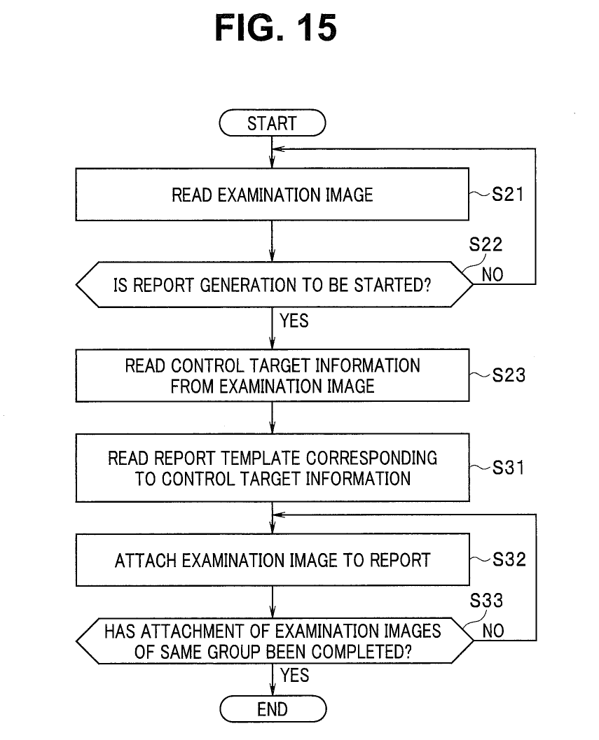

Next, a specific operation and the like of a report generating process of the endoscope apparatus 1 of the modification of the second embodiment will be described with reference to FIG. 15. FIG. 15 is a flowchart showing an example of a flow of the report generating process. Note that, in FIG. 15, processes similar to processes of FIG. 10 are given same reference numerals, and description will be omitted.

When reading control target information at step S23, the report generating portion 38A proceeds to step S31 and reads a report template corresponding to the control target information. Then, at step S32, the report generating portion 38A attaches the examination image to the report template.

At step S33, the report generating portion 38A judges whether attachment of examination images of a same group has been completed or not. If judging that attachment of the examination images of the same group has not been completed, the report generating portion 38A proceeds to step S32 and repeats a similar process. On the other hand, if judging that attachment of the examination images of the same group has been completed, the report generating portion 38A ends the report generating process.

By the report generating process as described above, the reports 201 to 203 shown in FIGS. 11 to 13 can be generated similarly to the second embodiment. Since reference information is shown in a report template in advance, the user can easily confirm that an examination has been correctly performed.

Note that, as for the steps in each flowchart in the present specification, execution order may be changed, a plurality of steps may be simultaneously executed, or the steps may be executed in different order for each execution, unless contrary to the nature of the steps.

The present invention is not limited to the embodiments and modification described above, and various changes, alterations and the like are possible within a range not departing from the spirit of the present invention.

What is claimed is:

1. An endoscope apparatus comprising:
a processor comprising hardware, wherein the processor is configured to acquire information about an object that can be controlled by a rotation assisting tool configured to cause an object including a rotating body to rotate, from the rotation assisting tool; and
a first storage configured to store reference information corresponding to the information about the object acquired by the processor.

2. The endoscope apparatus according to claim 1, wherein the processor generates a report with an image of the object picked up by an image sensor and the reference information corresponding to the information about the object acquired by the processor arranged in a same report.

3. The endoscope apparatus according to claim 2, wherein the processor generates a report with the image of the object picked up by the image sensor and a reference image included in the reference information arranged in the same report.

4. The endoscope apparatus according to claim 1, wherein the processor stores an image of the object picked up by an image sensor and the reference information corresponding to the information about the object acquired by the processor as one image.

5. The endoscope apparatus according to claim 1, wherein the processor acquires images corresponding to a circumference of the object which is the rotating body.

6. The endoscope apparatus according to claim 5, wherein the processor stores the images corresponding to the circumference of the object into a same folder.

7. The endoscope apparatus according to claim 6, wherein the processor generates a report with the images corresponding to the circumference of the object and the reference information corresponding to the information about the object acquired by the processor arranged in a same report.

8. The endoscope apparatus according to claim 6, wherein the processor generates a report using a template file corresponding to the acquired information about the object.

9. The endoscope apparatus according to claim 1, wherein the processor stores an image of an object including a first rotating body and an image of an object including a second rotating body into different folders.

10. The endoscope apparatus according to claim 1, wherein by acquiring information about a currently connected object from the rotation assisting tool, the processor determines whether the object is fitted to the rotation assisting tool or not.

11. The endoscope apparatus according to claim 10, wherein the processor determines that the object is fitted to the rotation assisting tool if the information about the object can be acquired from the rotation assisting tool and determines that the object is detached from the rotation assisting tool if the information about the object cannot be acquired from the rotation assisting tool.

12. The endoscope apparatus according to claim 11, wherein if the information about the object changes, the processor changes a folder for storing an image of the object.

13. An endoscope system comprising:
the endoscope apparatus according to claim 1; and
the rotation assisting tool according to claim 1.

14. A report generation method comprising:
acquiring information about an object that can be controlled by a rotation assisting tool configured to cause an object including a rotating body to rotate, from the rotation assisting tool; and
generating a report with an image of the object picked up by an image sensor and reference information corresponding to the acquired information about the object arranged in a same report.

15. The report generation method according to claim 14, wherein a report with the image of the object picked up by the image sensor and a reference image included in the reference information arranged in the same report is generated.

16. The report generation method according to claim 14, wherein images corresponding to a circumference of the object which is the rotating body are acquired; and
a report with the acquired images corresponding to the circumference of the object and the reference information corresponding to the acquired information about the object arranged in the same report is generated.

17. The report generation method according to claim 14, wherein a report is generated using a template file corresponding to the acquired information about the object.
